(19) United States
(12) Patent Application Publication
(10) Pub. No.: US 2024/0025140 A1
(43) Pub. Date: Jan. 25, 2024

(54) METHOD TO LAMINATE ePTFE TO A NITINOL BRAID WITH A SMOOTH OUTER DIAMETER

(71) Applicant: ABIOMED, Inc., Danvers, MA (US)

(72) Inventor: Christopher Nason Korkuch, Danvers, MA (US)

(73) Assignee: ABIOMED, Inc., Danvers, MA (US)

(21) Appl. No.: 18/224,644

(22) Filed: Jul. 21, 2023

Related U.S. Application Data
(60) Provisional application No. 63/391,384, filed on Jul. 22, 2022.

(51) Int. Cl. B32B 1/08; B32B 27/32; B32B 27/40; B32B 15/02; B32B 15/085; B32B 15/095; B32B 37/10

(52) U.S. Cl. CPC B32B 1/08; B32B 27/322; B32B 27/40; B32B 15/02; B32B 15/085; B32B 15/095; B32B 37/10; B32B 2307/536; B32B 2535/00

(57) ABSTRACT

A method to laminate ePTFE to Nitinol structures, such as an expandable sheath. Initially, the nitinol braid is slid into an outer ePTFE liner. An inner ePTFE liner is slid into the nitinol braid. The assembly of nitinol braid and inner and outer ePTFE liners is slid onto an inflatable silicone tube. The assembly inserted onto the silicone tube is placed in a heated die. The silicone tube is pressurized, causing it to inflate, forcing the inner ePTFE liner into contact with an inner surface of the nitinol structure. The die is cooled. The silicone tube is depressurized. The laminated structure is then removed from the die and the inflatable silicone tube.

### CROSS-REFERENCE TO RELATED APPLICATION

This application claims priority to and the benefit of U.S. Provisional Application No. 63/391,384, which was filed on Jul. 22, 2022, and is incorporated by reference herein.

### TECHNICAL FIELD

Described is a method to attach ePTFE to nitinol structures, such as an expandable sheath.

### BACKGROUND

Intracardiac heart pump assemblies can be introduced into the heart either surgically or percutaneously and used to deliver blood from one location in the heart or circulatory system to another location in the heart or circulatory system. For example, when deployed in the heart, an intracardiac pump can pump blood from the left ventricle of the heart into the aorta, or pump blood from the inferior vena cava into the pulmonary artery. Intracardiac pumps can be powered by a motor located outside of the patient's body (and accompanying drive cable) or by an onboard motor located inside the patient's body. Some intracardiac blood pump systems can operate in parallel with the native heart to supplement cardiac output and partially or fully unload components of the heart. Examples of such systems include the IMPELLA® family of devices (Abiomed, Inc., Danvers Mass.).

In one common approach, an intracardiac blood pump is inserted by a catheterization procedure through the femoral artery using an introducer sheath, which may be a peel away introducer sheath. The sheath may alternatively be inserted in other locations such as in the femoral vein or any path for delivery of a pump for supporting either the left or right side of the heart.

The introducer sheath may be inserted into the femoral artery through an arteriotomy to create an insertion path for the pump assembly. A portion of the pump assembly is then advanced through an inner lumen of the introducer sheath and into the artery. The requisite size of the arteriotomy is a matter of intense interest. Accordingly, expandable introducer sheaths have been developed so that a smaller arteriotomy opening is required to accommodate the sheath and the medical device passed therethrough. Accordingly, improvements in expandable introducer sheaths continue to be sought.

### BRIEF SUMMARY

Described herein is a method for laminating a liner of expanded polytetrafluoroethylene (ePTFE) on a surface of an expandable tube that may be made from nitinol or braided nitinol. Both ePTFE and nitinol are well known materials and are not described in detail herein. The method deploys an inflatable silicone tube that, when inflated brings one of inner ePTFE liner and/or an outer ePTFE liner into contact with a heated die that laminates the ePTFE liner against the expandable tube.

In one aspect a method for making an expandable sheath is described. According to one aspect of the method, an expandable tubular structure is placed within an outer liner. An inner liner is inserted within the expandable tubular structure. The expandable tubular structure having inner and outer liners is placed over an inflatable silicone tube. The inflatable silicone tube is placed within a heated die, which is either between the inflatable silicone tube and the inner liner or outside the outer liner. The silicone tube is inflated, thereby laminating the inner and outer liners to the expandable tubular structure by bringing one of the inner liner or the outer liner into contact with the heated tube. The heated die is then cooled and the silicone tube is depressurized, causing it to deflate. The laminated expandable sheath is removed from the die and the inflatable silicone tube.

In one aspect, each of the inner and outer liners is an expanded polytetrafluoroethylene (ePTFE). In a further aspect, a thermoplastic polyurethane is applied to an inner surface of the outer liner or an outer surface of the inner liner.

In one aspect, the expandable tubular structure is made from braided nitinol wires.

In a further aspect, pressuring the inflatable silicone tube forces the inner liner and outer liner into contact with the inner surface of the braid structure and an inner surface of the heated die, respectively. In one aspect of the above, the inflatable silicone tube has a free outer diameter that is less than an inner diameter of the inner liner. In a further aspect, the inflatable silicone tube is configured to expand to an outer diameter that brings one of the inner or outer liners into contact with the heated die. In one aspect, the inflatable silicone tube is located inside the inner liner.

In one aspect, the heated die is located outside of the outer liner. In an alternative aspect, the heated die is located inside of the inner liner. In one aspect, the heated die is brought to a temperature of 110° C. In one aspect, the inflatable silicone tube has a soft durometer of about 10 Shore A to about 50 Shore A. In one aspect, the inner liner of the laminated expandable sheath has an irregular surface that conforms to contours of the braid structure. In another aspect, the outer liner of the laminated expandable sheath has a smooth surface. In a further aspect, the laminated expandable sheath includes the inner liner, the outer liner, and the expandable tubular frame structure between the inner and outer liner.

Also described is an expandable laminated sheath having an expandable tubular frame structure having an inner surface and an outer surface and an expanded polytetrafluoroethylene inner liner laminated to the inner surface of the nitinol expandable braided tube structure and an expanded polytetrafluoroethylene outer liner laminated to the outer surface of the nitinol expandable braided tube structure, wherein the expandable laminated sheath is formed by inserting an inflatable silicone tube inside the inner liner and inflating the silicone tube until either the inner surface of the inner liner or the outer surface of the outer liner are brought into contact with a heated die.

In one aspect, one of either the outer surface of the inner liner or the inner surface of the outer liner is coated with a thermoplastic polyurethane. In one aspect, the expandable tubular frame is a nitinol braid structure. In one aspect, the inner and outer liners are formed from expanded polytetrafluoroethylene (ePTFE).

### BRIEF DESCRIPTION OF DRAWINGS

Aspects of what is described are illustrated in the drawings.

FIG. 1 illustrates a cross-section of a nitinol structure with inner and outer ePTFE liners.

FIG. 2 illustrates a cross-section of a heated die according to one aspect of the technology.

FIG. 3 illustrates a cross-section of a silicone tube according to one aspect of the technology.

FIG. 4 illustrates an assembly of the structures illustrated in FIGS. 1-3 that may be used to fabricate an ePTFE laminated nitinol structure.

FIG. 5 illustrates how the silicone tube is used to laminate the ePTFE liner to the nitinol braid in one aspect of the technology.

FIG. 6 illustrates how the inner ePTFE liner is laminated to the inner diameter of the nitinol braid while the outer ePTFE liner remains smooth according to one aspect of the technology.

FIG. 7 illustrates how the outer ePTFE liner is laminated to the outer diameter of the nitinol braid while the inner ePTFE liner remains smooth according to one aspect of the technology.

FIG. 8 is a flow chart according to one aspect of the method described herein.

### DETAILED DESCRIPTION

Embodiments of the present disclosure are described in detail with reference to the drawing figures wherein like reference numerals identify similar or identical elements. It is to be understood that the disclosed embodiments are merely examples of the disclosure, which may be embodied in various forms. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

In one aspect, the ePTFE liner is attached to the interior surface of the tubular frame. In another aspect, a primer is formed between the ePTFE liner and an inner surface of the lumen defined by the tubular frame. In another aspect, the introducer sheath assembly has an outer ePTFE liner formed on an exterior surface of the tubular frame. In another aspect, the introducer sheath assembly has inner and an outer ePTFE liners formed on both an interior surface and an exterior surface of the tubular frame. The tubular frame may be made of braided nitinol wires. Nitinol is a well-known alloy of nickel and titanium, where the nickel and titanium are in roughly equal percentages.

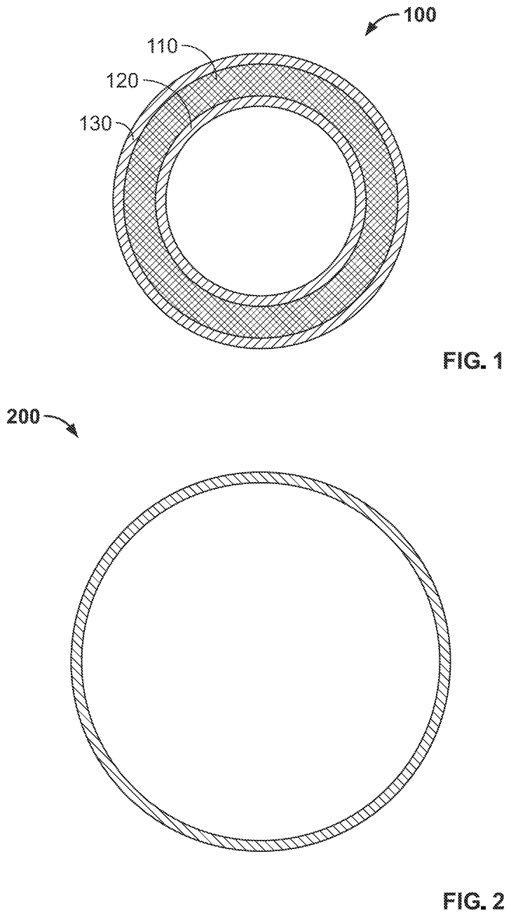

With reference to FIG. 1, an expandable sheath structure 100 that has a nitinol structure 110 with an inner liner of ePTFE 120 and an outer liner of ePTFE 130 is illustrated. Each of the nitinol structure 110, the inner ePTFE liner 120, and the outer ePTFE liner 130 is a tube.

In one aspect, the nitinol structure is a braided nitinol wire structure with the following features, which are offered by way of example and not by way of limitation.
i. The nitinol structure has an outer diameter (OD) of about 3.9 to about 4.1 mm; the nitinol structure is formed from hard nitinol wire braid of about 75 μm to about 125 μm in diameter. The nitinol may be SE508 nitinol. In one aspect, the braids are brought or wound together in a 48-end, 2×2 braid pattern, using an active temperature (Af) of about 10° C. to about under a pressure of about 16.5 PPI to about 17.6 PPI (pounds per square inch) on about 4.15 mm pins, with a braid angle of about 34° to about 40° included; and
ii. A thermoplastic polyurethane (TPU) primer (amount applied is about 20 mg to about 60 mg for a 150 cm long nitinol braid). In one example, the hardness is about 75 shore A, having a specific gravity of about 1.1 to about 1.4 SG, and a tensile strength @ 50% elongation is about 100 psi to about 650 psi. The ultimate elongation for the material is about 350% to about 750%. The properties of nitinol and polyurethane are well known and these are examples of properties for these materials that are suitable for the claimed structures. About, as used herein, is ±10% of the values expressed.

Each of the inner and outer ePTFE liners has the following features, which are offered by way of example and not by way of limitation.
i. Each ePTFE liner has an inner diameter (ID) of about 3.9 mm to about 4.1 mm ID; each ePTFE liner has a thickness of about 0.040 mm to about 0.060 mm, with a density of about 0.4 g/cc and a tensile strength at 50% elongation is about 0.15 to about 0.45 N/mm; and
ii. TPU OD Seal (amount applied may be about 30 mg to about 75 mg for a 150 cm long ePTFE liner). In one example, the hardness may be about 42-73 shore A, having a specific gravity of about 0.96 SG to about 1.04 SG, and a tensile strength at 50% elongation may be about 130 psi to about 455 psi. The ultimate elongation for the material may be about 500% to about 626%.

The properties of ePTFE are well known and these are examples of properties for the materials that are suitable for the claimed structures.

Referring to FIG. 2, the figure illustrates a heated die 200 that defines an inner diameter that approximately equal to the target final outer diameter of the expandable sheath with inner and outer ePTFE liners 120, 130 laminated on a nitinol structure. In one aspect, the target final outer diameter is from about 4.5 mm to about 4.6 mm.

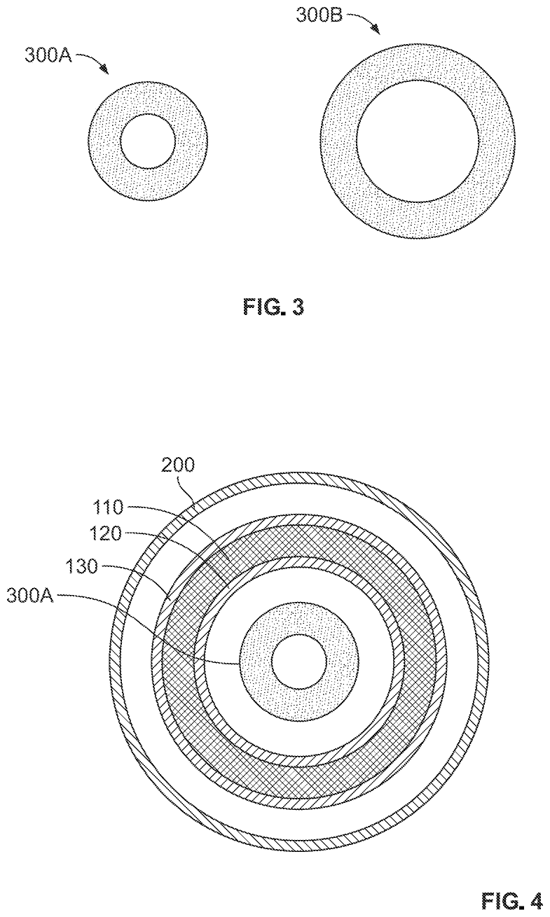

Referring to FIG. 3, the figure illustrates a silicone tube 300A with a free outer diameter that is less than an inner diameter of the inner ePTFE liner 120 but is configured to expand with internal pressure to an outer diameter 300B that is greater than the diameter of the inner ePTFE liner 120. Free outer diameter, as used herein, is an outer diameter when the silicone tube is in the uninflated state. Preferably, the inflatable silicone tube has an outer diameter (OD) of about 3.0 mm to about 3.8 mm and an inner diameter (ID) of about 1.0 mm to about 3.3 mm. In one example, the hardness is about 10 shore A to about 50 shore A and the internal pressure of the inflatable silicone tube is about 1.6 psi to about 7 psi. In one aspect, the heated die 200 is configured to reach about 90° C. to about 150° C. in about 2-20 min. The heated die 200 is cooled off with “heat off” for about 5 to about 20 minutes, with air flow for about 1 to about 10 minutes, and with water cooling for about 1 to about 5 min.

Referring to FIG. 4, there is illustrated the assembly of the inner ePTFE inner liner 120, and the outer ePTFE liner 130 with the nitinol braid 110 in between. The inflatable silicone tube 300A is located inside the inner ePTFE liner 120 and the heated die 200 is located on the exterior of the outer ePTFE outer liner 130. According to the method, the heated die 200 is brought to a temperature of 110° C., and the nitinol structure (with inner and outer ePTFE liners) is loaded onto the inflatable silicone tube and then slid into the heated die 200.

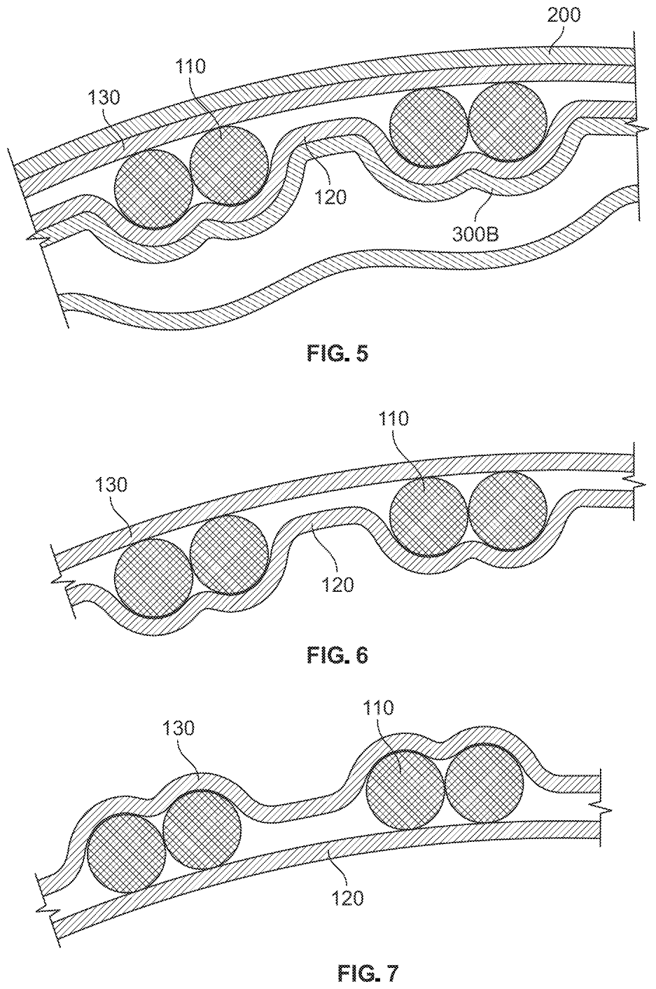

Referring to FIG. 5, there is illustrated a partial cross-section of the assembly illustrated in FIG. 4 with the silicone tube 300B having been inflated. The structure (nitinol structure 110 between the inner and outer ePTFE liners 120 and 130) is pressed against the inner surface of the heated die 200. Because the silicone has a soft durometer (e.g., shore 10 A to about 50 A), the silicone tube 300B will press up into the nitinol braid 110, laminating the inner ePTFE liner 120 to the inner diameter of the nitinol braid structure 110.

Referring to FIG. 6, illustrated is a partial cross-section of the assembly after being removed from the tool (the tool being the heated die and the inflatable silicone tube). The assembly is removed by deflating (depressurizing) the inflatable silicone tube. The inner ePTFE liner 120 has an irregular surface that conforms to the contours of the braided nitinol structure 110. The outer ePTFE liner 130 has a smooth surface. The inner surface of the assembly is flushed with saline in use. As such the inner surface may not pose a prolonged thrombogenicity risk due to flow dynamics. The inner surface also has reduced surface area contact with devices being passed through the structure, which lowers the insertion force required for those devices.

FIG. 7 is an alternative aspect of what is illustrated in FIG. 6. The structure in FIG. 7 is fabricated by providing the heated die (not shown) inside the inner diameter of the inner ePTFE liner 120. The inflatable silicone tube is placed on the outer diameter of the structure and will apply pressure to the outer ePTFE liner 130 when inflated. The structure that results has a smooth inner ePTFE liner 120 and a conformal outer ePTFE liner 130. The textured outer ePTFE-coated diameter poses a risk of thrombogenicity through low surface shear rate, low velocity, and areas of recirculation of blood.

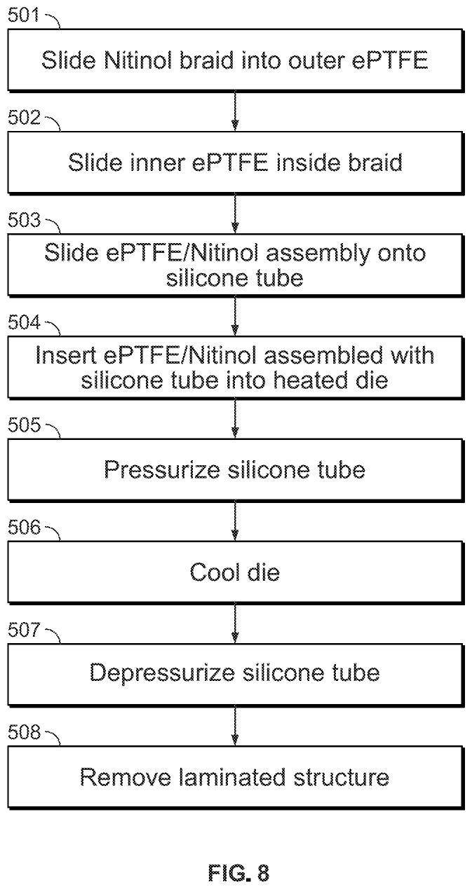

FIG. 8 describes the method for making the expandable sheath structure described herein. Initially, the nitinol braid structure is slid or placed into an outer ePTFE liner in step 501. In step 502, an inner ePTFE liner is slid or placed into the nitinol braid structure. In step 503, the assembly of nitinol braid structure and inner and outer ePTFE liners is slid or inserted onto the inflatable silicone tube such that the inflatable silicone tube is positioned within the lumen of the assembly. In step 504, the assembly inserted onto the silicone tube is placed in the heated die such that the heated die surrounds the assembly. In step 505, the inflatable silicone tube is pressurized, causing it to inflate, forcing the inner ePTFE liner into contact with an inner surface of the nitinol braid structure and pressing the nitinol braid structure to force the outer ePTFE liner into contact with and press against an inner surface of the heated die 200. Thus, pressurizing the inflatable silicone tube allows the inner ePTFE liner and the outer ePTFE liner to be laminated to an inner surface of the braid structure and an outer surface of the braid structure, respectively, to form the expandable sheath structure. In step 506, the heated die is cooled. In step 507, the silicone tube is depressurized, causing the inflatable silicone tube to deflate. In step 508, the laminated expandable sheath structure is removed from the die and the inflatable silicone tube.

From the foregoing and with reference to the various figure drawings, those skilled in the art will appreciate that certain modifications can also be made to the present disclosure without departing from the scope of the same. While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

1. A method for making an expandable sheath, the method comprising:
sliding an expandable tubular structure into an outer liner;
sliding an inner liner into the expandable tubular structure;
inserting an assembly of the expandable tubular structure, inner liner, and outer liner onto an inflatable silicone tube;
placing the assembly with the inflatable silicone tube in a heated die;
pressuring the inflatable silicone tube, causing the inflatable silicone tube to inflate, to laminate the inner liner and the outer liner to an inner surface of the expandable tubular structure and an outer surface of the expandable tubular structure, respectively, to form the expandable sheath;
cooling the heated die;
depressurizing the silicone tube, thereby causing the inflatable silicone tube to deflate; and
removing the laminated expandable sheath from the die and the inflatable silicone tube.

2. The method of claim 1, wherein each of the inner and outer liners is an expanded polytetrafluoroethylene (ePTFE).

3. The method of claim 2, wherein a thermoplastic polyurethane is applied to an inner surface of the outer liner.

4. The method of claim 2, wherein a thermoplastic polyurethane is applied to an outer surface of the inner liner.

5. The method of claim 1, wherein the expandable tubular structure is made from braided nitinol wires.

6. The method of claim 1, wherein pressuring the inflatable silicone tube forces the inner liner and outer liner into contact with the inner surface of the expandable tubular structure and an inner surface of the heated die, respectively.

7. The method of claim 1, wherein the inflatable silicone tube has a free outer diameter that is less than an inner diameter of the inner liner.

8. The method of claim 7, wherein the inflatable silicone tube is configured to expand to an outer diameter that brings one of the inner liner or the outer liner into contact with the heated die.

9. The method of claim 1, wherein the inflatable silicone tube is located inside the inner liner.

10. The method of claim 8, wherein the heated die is located outside of the outer liner.

11. The method of claim 8, wherein the heated die is located inside of the inner liner.

12. The method of claim 1, wherein the heated die is brought to a temperature of 110° C.

13. The method of claim 1, wherein the inflatable silicone tube has a soft durometer of about 10 Shore A to about 50 Shore A.

14. The method of claim 1, wherein the inner liner of the laminated expandable sheath has an irregular surface that conforms to contours of the expandable tubular structure.

15. The method of claim 1, wherein the outer liner of the laminated expandable sheath has a smooth surface.

16. The method of claim 1, wherein the laminated expandable sheath includes the inner liner, the outer liner, and the expandable tubular structure between the inner and outer liner.

17. An expandable laminated sheath comprising:
an expandable tubular frame structure having an inner surface and an outer surface;
an expanded polytetrafluoroethylene inner liner laminated to the inner surface of the nitinol expandable tubular frame structure;
an expanded polytetrafluoroethylene outer liner laminated to the outer surface of the expandable tubular structure; and
wherein the expandable laminated sheath is formed by inserting an inflatable silicone tube inside the inner liner and inflating the silicone tube until either the inner surface of the inner liner or the outer surface of the outer liner are brought into contact with a heated die.

18. The expandable laminated sheath of claim 17, wherein one of either the outer surface of the inner liner or the inner surface of the outer liner is coated with a thermoplastic polyurethane.

19. The expandable laminated sheath of claim 17, wherein the expandable tubular frame is a nitinol structure formed from braided nitinol wires.

20. The expandable laminated sheath of claim 17, wherein the inner and outer liners are formed from expanded polytetrafluoroethylene (ePTFE).
